SEQUENCE LISTING (1 sequence)

SEQ ID NO: 1 - DNA, 20 bp, synthetic construct
  source 1..20: mol_type = other DNA
gcagaggtgaagcgaagtgc

(19) United States
(12) Patent Application Publication
(10) Pub. No.: US 2025/0092401 A1
(43) Pub. Date: Mar. 20, 2025

(54) COMBINATION THERAPY FOR TREATING HEPATITIS B VIRUS INFECTIONS

(71) Applicant: GLAXOSMITHKLINE INTELLECTUAL PROPERTY DEVELOPMENT LIMITED, Stevenage (GB)

(72) Inventors: Jared Loren DELAHAYE, Collegeville, PA (US); Martin R. LEIVERS, Collegeville, PA (US); Malek OKOUR, Narberth, PA (US); Shihyun Kieffer YOU, Phoenixville, PA (US)

(21) Appl. No.: 18/832,756

(22) PCT Filed: Jan. 26, 2023
(86) PCT No.: PCT/IB2023/050687

§ 371 Date: Jul. 24, 2024

Related U.S. Application Data
(60) Provisional application No. 63/304,091, filed on Jan. 28, 2022.

(51) Int. Cl. C12N 15/113; A61K 31/4375; A61K 45/06; A61P 31/20

(52) U.S. Cl. CPC C12N 15/1131; A61K 31/4375; A61K 45/06; A61P 31/20; C12N 2310/11; C12N 2310/315; C12N 2310/321; C12N 2310/3341; C12N 2310/341

(57) ABSTRACT

The present disclosure is related to methods for treating hepatitis B infections. The methods comprising administering a subject in need thereof a PAPD5/7 inhibitor and a modified ASO that targets HBV mRNAs.

### FIELD

The present disclosure relates to methods for treating hepatitis B virus (HBV) infections. These methods comprise administering to a subject in need thereof an RNA polymerase associated domain containing proteins 5 and 7 (PAPD 5/7) inhibitor and administering a modified antisense oligonucleotide (ASO).

### BACKGROUND

Hepatitis B virus (HBV) is a strict hepatotropic, double-stranded DNA containing virus. Although DNA is the genetic material, the replication cycle involves a reverse transcription step to copy a pregenomic RNA into DNA. Primary infection with HBV causes an acute hepatitis with symptoms of organ inflammation, fever, jaundice, and increased liver transaminases in blood. Those patients that are not able to overcome the virus infection suffer a chronic disease progression over many years with increased risk of developing cirrhotic liver or liver cancer.

HBV infection results in the production of two different particles: 1) the HBV virus itself (or Dane particle) which includes a viral capsid assembled from the HBV core antigen protein (HBcAg) and is covered by the hepatitis B surface antigen (HBsAg) and is capable of reinfecting cells and 2) subviral particles (or SVPs) which are high density lipoprotein-like particles comprised of lipids, cholesterol, cholesterol esters and the small and medium forms of the hepatitis B surface antigen (HBsAg) which are non-infectious. For each viral particle produced, 1,000-10,000 SVPs are released into the blood. As such SVPs (and the HBsAg protein they carry) represent the overwhelming majority of viral protein in the blood. HBV infected cells also secrete a soluble proteolytic product of the pre-core protein called the HBV e-antigen (HBeAg).

Currently the recommended therapies for chronic HBV infection by the American Association for the Study of Liver Diseases (AASLD) and the European Association for the Study of the Liver (EASL) include interferon alpha (INFα), pegylated interferon alpha-2a (Peg-IFN2α), entecavir, and tenofovir. However, typical interferon therapy is 48-weeks and results in serious and unpleasant side effects, and HBeAg seroconversion, 24 weeks after therapy has ceased, ranges from only 27-36%. Seroconversion of HBsAg is even lower—only 3% observed immediately after treatment ceases, with an increase to upwards of 12% after 5 years.

The nucleoside and nucleotide therapies entecavir and tenofovir have been successful at reducing viral load, but the rates of HBeAg seroconversion and HBsAg loss are even lower than those obtained using IFNα therapy. Other similar therapies, including lamivudine (3TC), telbivudine (LdT), and adefovir are also used, but for nucleoside/nucleotide therapies in general, the emergence of resistance limits therapeutic efficacy.

Thus, there is a need to discover and develop new anti-HBV therapies, more particularly, therapies capable of increasing HBeAg and/or HBsAg seroconversion rates.

### SUMMARY

In one aspect, the present disclosure provides a method for treating a hepatitis B virus (HBV) infection in a subject in need thereof, the method comprising administering to the subject a therapeutically effective amount of Compound A having the structure:

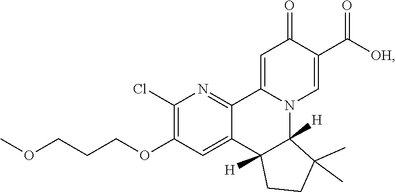 [SMILES: COCCCOc1cc2c(nc1Cl)-c1cc(=O)c(C(=O)O)cn1[C@H]1[C@@H]2CCC1(C)C]

or a pharmaceutically acceptable salt thereof, and
   administering to the subject a therapeutically effective amount of a single-stranded modified oligonucleotide including 20 linked nucleosides having a nucleobase sequence of 5′-GCAGAGGTGAAGCGAAGTGC-3′ (SEQ ID NO:1), wherein the modified oligonucleotide includes:
   a gap segment consisting of ten linked deoxynucleosides,
   a 5′ wing segment consisting of 5 linked nucleosides, and
   a 3′ wing segment consisting of 5 linked nucleosides,
wherein the gap segment is positioned between the 5′ wing segment and the 3′ wing segment, wherein each nucleoside of each wing segment includes a 2′-O-methoxyethyl sugar, wherein each internucleoside linkage is a phosphorothioate linkage, and wherein each cytosine is a 5-methylcytosine (see FIG. 1).

Compound A described herein is a PAPD5/7 inhibitor, disclosed as Compound No. 220 in WO2019/069293, which is incorporated herein by reference in its entirety.

The single-stranded modified oligonucleotide described herein is an antisense oligonucleotide (ASO) as disclosed in WO2012/145697, which is incorporated herein by reference in its entirety. In one embodiment, the ASO is compound ISIS No. 505358 as disclosed in WO2012/145697, and it is also referred to as bepirovirsen.

In one embodiment, the present disclosure provides a method for treating chronic hepatitis B (CHB) in a human in need thereof, the method comprising administering to the subject a therapeutically effective amount of Compound A and a therapeutically effective amount of bepirovirsen.

In another aspect, the present disclosure provides a method for reducing serum HBsAg levels in a subject in need thereof, the method comprising administering to the subject a therapeutically effective amount of Compound A, or a pharmaceutically acceptable salt thereof, and administering to the subject a therapeutically effective amount of the single-stranded modified oligonucleotide as described herein.

In another aspect, the present disclosure provides a method for reducing serum HBV DNA levels in a subject in need thereof, the method comprising administering to the subject a therapeutically effective amount of Compound A, or a pharmaceutically acceptable salt thereof, and administering to the subject a therapeutically effective amount of the single-stranded modified oligonucleotide as described herein.

In another aspect, there is provided a combination for use in the treatment of chronic hepatitis B, comprising Compound A having the structure:

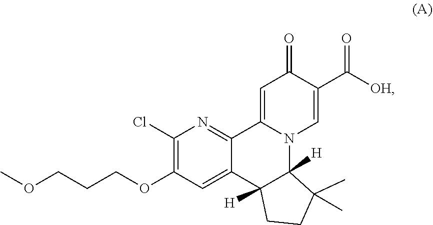 [SMILES: COCCCOc1cc2c(nc1Cl)-c1cc(=O)c(C(=O)O)cn1[C@H]1[C@@H]2CCC1(C)C]

or a pharmaceutically acceptable salt thereof, and
a single-stranded modified oligonucleotide comprising 20 linked nucleosides having a nucleobase sequence of SEQ ID NO:1, wherein the modified oligonucleotide comprises:
a gap segment consisting of ten linked deoxynucleosides,
a 5′ wing segment consisting of 5 linked nucleosides, and
a 3′ wing segment consisting of 5 linked nucleosides,
   wherein the gap segment is positioned between the 5′ wing segment and the 3′ wing segment, wherein each nucleoside of each wing segment comprises a 2′-O-methoxyethyl sugar, wherein each internucleoside linkage is a phosphorothioate linkage, and wherein each cytosine is a 5-methylcytosine.

In one embodiment, the combination is for use in a method of treating chronic hepatitis B in a subject, wherein the method comprises administering Compound A and the modified oligonucleotide concomitantly to the subject.

In one embodiment, the combination is for use in a method of treating chronic hepatitis B (CHB) in a human in need thereof, wherein the method comprises administering to the subject a therapeutically effective amount of Compound A and a therapeutically effective amount of bepirovirsen.

In another embodiment, the combination is for use in a method of reducing serum HBsAg levels in a subject in need thereof, the method comprising administering to the subject a therapeutically effective amount of Compound A, or a pharmaceutically acceptable salt thereof, and administering to the subject a therapeutically effective amount of the single-stranded modified oligonucleotide as described herein.

In a further embodiment, the combination is for use in a method of reducing serum HBV DNA levels in a subject in need thereof, the method comprising administering to the subject a therapeutically effective amount of Compound A, or a pharmaceutically acceptable salt thereof, and administering to the subject a therapeutically effective amount of the single-stranded modified oligonucleotide as described herein.

### BRIEF DESCRIPTION OF THE DRAWINGS

FIG. 1 depicts the structure of chimeric 2′-MOE phosphorothioate oligonucleotides (MOE Gapmer).

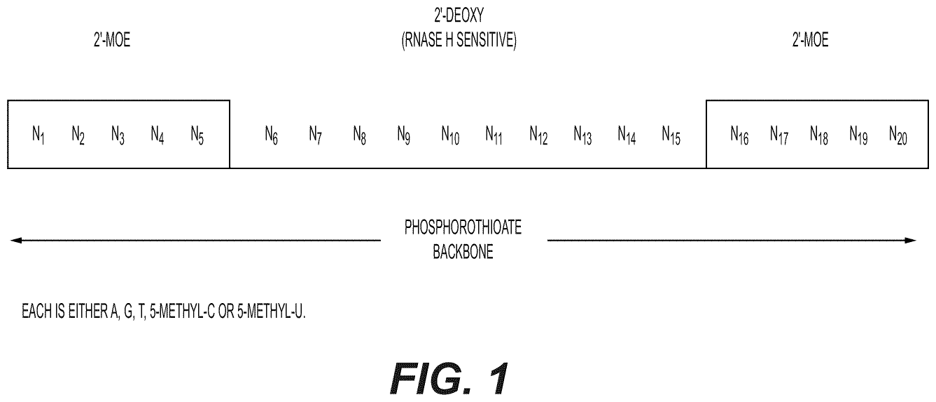

FIG. 2 depicts enzymatic activity of PAPD5 and PAPD7 in biochemical assay for Compound A and RG7834.

FIG. 3 depicts the effect of monotreatment with Compound A on HBsAg levels in AAV-HBV mice.

FIG. 4 depicts the effect of concurrent treatment with Compound A and bepirovirsen on HBsAg levels in AAV-HBV mice.

FIG. 5 depicts the effect of sequential treatment with Compound A and bepirovirsen on HBsAg levels in AAV-HBV mice.

### DETAILED DESCRIPTION

### Definitions

Unless defined otherwise, all technical and scientific terms used herein have the same meanings as commonly understood by one of ordinary skill in the art. For example, certain terms used herein are defined as described in “A multilingual glossary of biotechnological terms: (IUPAC Recommendations),” Leuenberger, H. G. W, Nagel, B. and Klbl, H. eds. (1995), Helvetica Chimica Acta, CH-4010 Basel, Switzerland).

Throughout this specification and the claims which follow, unless the context requires otherwise, the word “comprise,” and variations such as “comprises” and “comprising,” will be understood to imply the inclusion of a stated element, integer, or step, or group of elements, integers, or steps but not the exclusion of any other element, integer, or step, or group of elements, integers, or steps.

Unless otherwise indicated, the following terms have the following meanings:

“2′-O-methoxyethyl” (also 2′-MOE and 2′-O(CH₂)₂—OCH₃) refers to an O-methoxyethyl modification at the 2′ position of a furanose ring. A 2′-O-methoxyethyl modified sugar is a modified sugar.

“2′-O-methoxyethyl nucleoside” (also 2′-MOE nucleoside) refers to a nucleoside comprising a 2′-O-methoxyethyl modified sugar moiety.

“5-methylcytosine” refers to a cytosine modified with a methyl group attached to the 5-position. A 5-methylcytosine is a modified nucleobase.

“About,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass±10% of the recited value and the range is included.

“Active pharmaceutical agent” refers to the substance or substances in a pharmaceutical composition that provide a therapeutic benefit when administered to a subject. For example, in some embodiments, an antisense oligonucleotide targeted to HBV is an active pharmaceutical agent. In some embodiments, a PAPD5/7 inhibitor is an active pharmaceutical agent.

“Administered concomitantly” (or concurrent administration) refers to the co-administration of two active pharmaceutical agents to a subject during a time period in any manner in which the first administered active pharmaceutical agent is still present in the subject in a therapeutically effective amount when the second active pharmaceutical agent is administered. Concomitant administration does not require that both agents be administered in a single pharmaceutical composition, in the same dosage form, or by the same route of administration. The effects of both agents can be overlapping for a period of time and need not be coextensive.

“Administered sequentially” refers to administration of a first active pharmaceutical agent, followed by administration of a second active pharmaceutical agent a significant time later such that the first administered active pharmaceutical agent is not present in the subject in a therapeutically effective amount when the second active pharmaceutical agent is administered. The period of time between two sequential administrations may be between 1 week and 24 weeks, for example, between 2 weeks and 12 weeks, for example, 1 week, 2 weeks, 4 weeks, 6 weeks, 8 weeks, 10 weeks, or 12 weeks.

“Animal” refers to a human or non-human animal, including, but not limited to, mice, rats, rabbits, dogs, cats, pigs, and non-human primates, including, but not limited to, monkeys and chimpanzees.

“Antisense compound” refers to an oligomeric compound that is capable of undergoing hybridization to a target nucleic acid through hydrogen bonding. Examples of antisense compounds include single-stranded and double-stranded compounds, such as, antisense oligonucleotides, siRNAs, shRNAs, snoRNAs, miRNAs, and satellite repeats.

“Antisense oligonucleotide” refers to a single-stranded oligonucleotide having a nucleobase sequence that permits hybridization to a corresponding region or segment of a target nucleic acid.

“Chemically distinct region” refers to a region of an antisense compound that is in some way chemically different than another region of the same antisense compound. For example, a region having 2′-O-methoxyethyl nucleos(t)ides is chemically distinct from a region having nucleos(t)ides without 2′-O-methoxyethyl modifications.

“Chimeric antisense compound” refers to an antisense compound that has at least 2 chemically distinct regions, each region having a plurality of subunits.

“Chronic hepatitis B (CHB) infection” occurs when a person initially suffers from an acute infection but is then unable to fight off the infection. About 90% of infants infected at birth will progress to chronic disease. However, as a person ages, the risk of chronic infection decreases such that between 20%-50% of people infected as children and less than 10% of people infected as adults will progress from acute to chronic infection.

“Deoxyribonucleoside” refers to a nucleoside having a hydrogen at the 2′ position of the sugar portion of the nucleoside. Deoxyribonucleosides may be modified with any of a variety of substituents.

“Dose” refers to a specified quantity of a pharmaceutical agent provided in a single administration, or in a specified time period. In certain embodiments, a dose may be administered in two or more boluses, tablets, or injections. For example, in certain embodiments, where subcutaneous administration is desired, the desired dose requires a volume not easily accommodated by a single injection. In such embodiments, two or more injections may be used to achieve the desired dose. In certain embodiments, a dose may be administered in two or more injections to minimize injection site reaction in an individual.

“Dosing regimen” is a combination of doses designed to achieve one or more desired effects.

“Duration” refers to the period of time during which an activity or event continues. In certain embodiments, the duration of treatment is the period of time during which doses of a pharmaceutical agent are administered.

“Effective amount” in the context of modulating an activity or of treating or preventing a condition refers to the administration of that amount of an active ingredient to a subject in need of such modulation, treatment, or prophylaxis, either in a single dose or as part of a series, that is effective for modulation of that effect, or for treatment or prophylaxis or improvement of that condition.

“Gapmer” refers to a chimeric antisense compound in which an internal region having a plurality of nucleosides that support RNase H cleavage is positioned between external regions, wherein the nucleosides comprising the internal region are chemically distinct from the nucleosides comprising the external regions. The internal region may be referred to as the “gap,” “gap region,” or “gap segment,” and the external regions may be referred to as the “wings,” “wing regions,” or “wing segments.”

“HBV” refers to mammalian hepatitis B virus, including human hepatitis B virus. The term encompasses geographical genotypes of hepatitis B virus, particularly human hepatitis B virus, as well as variant strains of geographical genotypes of hepatitis B virus. Human geographical genotypes of HBV include genotypes: A (Northwest Europe, North America, Central America); B (Indonesia, China, Vietnam); C (East Asia, Korea, China, Japan, Polynesia, Vietnam); D (Mediterranean area, Middle East, India); E (Africa); F (Native Americans, Polynesia); G (United States, France); and H (Central America).

“HBV antigen” refers to any hepatitis B virus antigen or protein, including core proteins such as “hepatitis B core antigen” or “HBcAg,” “hepatitis B E antigen” or “HBeAg,” and envelope proteins such as “HBV surface antigen” or “HBsAg.”

“Hepatitis B E antigen” or “HBeAg” is a secreted, non-particulate form of HBV core protein. HBV antigens HBeAg and HBcAg share primary amino acid sequences, so show cross-reactivity at the T cell level. HBeAg is not required for viral assembly or replication, although studies suggest it may be required for establishment of chronic infection.

“HBV surface antigen” or “HBsAg” is the envelope protein of infectious HBV viral particles (Dane particles) but is also secreted as a non-infectious subviral particle (SVP) with serum levels 1000-fold higher than HBV viral particles. The serum levels of HBsAg in an infected person or animal can be as high as 1000 g/mL (Kann and Gehrlich (1998) Topley & Wilson's Microbiology and Microbial Infections, 9th ed. 745).

“Hepatitis B-related condition” or “HBV-related condition” refers to any disease, biological condition, medical condition, or event which is exacerbated, caused by, related to, associated with, or traceable to a hepatitis B infection, exposure, or illness. The term hepatitis B-related condition includes chronic HBV infection, inflammation, fibrosis, cirrhosis, liver cancer, serum hepatitis, jaundice, liver inflammation, liver fibrosis, liver failure, diffuse hepatocellular inflammatory disease, hemophagocytic syndrome, HBV viremia, liver disease related to transplantation, and conditions having symptoms which may include any or all of the following: flu-like illness, weakness, aches, headache, fever, loss of appetite, diarrhea, nausea and vomiting, pain over the liver area of the body, clay- or grey-colored stool, itching all over, and dark-colored urine, when coupled with a positive test for presence of a hepatitis B virus, a hepatitis B viral antigen, or a positive test for the presence of an antibody specific for a hepatitis B viral antigen.

“Induce,” “inhibit,” “potentiate,” “elevate,” “increase,” “decrease” or the like, generally denote quantitative differences between two states. Such terms may refer to a statistically significant difference between the two states. For example, “an amount effective to inhibit the activity or expression of HBV” refers to the level of activity or expression of HBV in a treated cell that is quantitatively different, and may be statistically significant, from the level of HBV activity or expression in untreated cells. Such terms are applied to, for example, levels of expression, and levels of activity.

“Internucleoside linkage” refers to the chemical bond between nucleosides. Phosphodiester and phosphorothioate are examples of internucleoside linkages.

“Linked nucleosides” refers to adjacent nucleosides linked together by an internucleoside linkage.

“Modified internucleoside linkage” refers to a substitution or any change from a naturally occurring internucleoside bond (i.e., a phosphodiester internucleoside bond), for example a phosphorothioate internucleoside bond.

“Modified nucleobase” refers to any nucleobase other than adenine, cytosine, guanine, thymidine, or uracil. An “unmodified nucleobase” refers to the purine bases adenine (A) and guanine (G), and the pyrimidine bases thymine (T), cytosine (C) and uracil (U). A modified nucleobase includes methylcytosine.

“Modified nucleoside” refers to a nucleoside having, independently, a modified sugar moiety and/or modified nucleobase.

“Modified nucleotide” refers to a nucleotide having, independently, a modified sugar moiety, modified internucleoside linkage, and/or modified nucleobase.

“Modified oligonucleotide” refers to an oligonucleotide comprising at least one modified internucleoside linkage, a modified sugar, and/or a modified nucleobase. A modified oligonucleotide may be, according to some embodiments of the present disclosure, a chimeric antisense compound including an internal region having a plurality of nucleosides that support RNase H cleavage, positioned between external regions, for example, between two external regions, such as a 5′ external region and a 3′ external region, each external region having one or more nucleosides, and wherein the nucleosides included in the internal region are chemically distinct from the nucleosides included in the external regions. A modified oligonucleotide can be in the form of a free acid or a pharmaceutically acceptable salt thereof (e.g. a sodium salt), or a mixture thereof.

“Nucleic acid” refers to molecules composed of monomeric nucleotides. A nucleic acid includes, but is not limited to, ribonucleic acids (RNA), deoxyribonucleic acids (DNA), single-stranded nucleic acids, double-stranded nucleic acids, small interfering ribonucleic acids (siRNA), and microRNAs (miRNA). Nucleic acids may include modified and/or unmodified nucleotides without limitation.

“Nucleobase” refers to a heterocyclic moiety capable of pairing with a base of another nucleic acid.

“Nucleobase sequence” refers to the order of contiguous nucleobases independent of any sugar, linkage, and/or nucleobase modification.

“Oligonucleotide” refers to a polymer of linked nucleosides, each of which can be modified or unmodified, independent one from another.

“Pharmaceutically acceptable salts” refer to physiologically and pharmaceutically acceptable salts of compounds, i.e., salts that retain the desired biological activity of the parent active ingredients and do not impart undesired toxicological effects thereto.

“Pharmaceutical agent” refers to a substance that provides a therapeutic benefit when administered to a subject. For example, a PAPD5/7 inhibitor, and/or an antisense oligonucleotide targeting HBV RNA, are pharmaceutical agents.

“Phosphorothioate linkage” refers to a linkage between nucleosides where the phosphodiester bond is modified by replacing one of the non-bridging oxygen atoms with a sulfur atom. A phosphorothioate linkage is a modified internucleoside linkage.

“Region” is defined as a portion of, for example, a nucleic acid having at least one identifiable structure, function, or characteristic.

“Segments” may refer to smaller, or sub-portions of, regions within, for example, a nucleic acid.

“Seroclearance” refers to HBsAg and/or HBV DNA levels below the lower limit of quantification (i.e. <LLOQ) in a CHB patient. In testing human samples, when serum HBsAg level is measured by a sandwich immunoassay with COBAS HBsAg quant II (Roche), the LLOQ is 0.05 IU/mL. In testing human samples, when the serum HBV DNA level is measured with COBAS Ampliprep/COBAS Taqman HBV test v.2.0 (Roche), the LLOQ is 20 IU/mL.

“Subject” refers to a human or non-human animal selected for treatment or therapy. In one embodiment, the subject is a human.

“Therapeutically effective amount” refers to the administration of an agent to a subject, either alone or as part of a pharmaceutical composition and either in a single dose or as part of a series of doses, in an amount capable of having any detectable, positive effect on any symptom, aspect, or characteristic of a disease or condition when administered to the subject.

“Treatment” refers to administering a composition to a subject to affect an alteration or improvement of the disease or condition. The term “treating” as used herein in relation to chronic hepatitis B infection refers to the administration of suitable compositions with the intention of reducing the symptoms of CHB, preventing the progression of CHB or reducing the level of one or more detectable markers of CHB.

### Methods

The present disclosure provides methods for treating a hepatitis B virus (HBV) infection. In some embodiments, the HBV infection is chronic hepatitis B (CHB).

In some embodiments, the methods for treating an HBV infection in a subject in need thereof comprises administering to the subject a therapeutically effective amount of a PAPD5/7 inhibitor and an antisense oligonucleotide.

In some embodiments, the method for treating an HBV infection in a subject in need thereof comprises administering to the subject a therapeutically effective amount of Compound A having the structure:

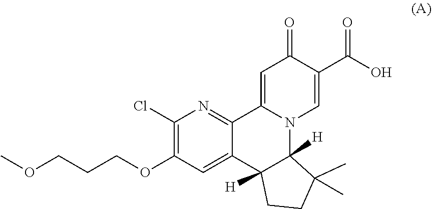 [SMILES: COCCCOc1cc2c(nc1Cl)-c1cc(=O)c(C(=O)O)cn1[C@H]1[C@@H]2CCC1(C)C]

or a pharmaceutically acceptable salt thereof.

In another embodiment, the method comprises administering to the subject a therapeutically effective amount of Compound A and administering to the subject a single-stranded modified oligonucleotide comprising 20 linked nucleosides and having a nucleobase sequence:

(SEQ ID NO: 1)
5′-GCAGAGGTGAAGCGAAGTGC-3′,

wherein the modified oligonucleotide comprises:
   a gap segment consisting of ten linked deoxynucleosides,
   a 5′ wing segment consisting of 5 linked nucleosides, and
   a 3′ wing segment consisting of 5 linked nucleosides,
wherein the gap segment is positioned between the 5′ wing segment and the 3′ wing segment, wherein each nucleoside of each wing segment comprises a 2′-O-methoxyethyl sugar, wherein each internucleoside linkage is a phosphorothioate linkage, and wherein each cytosine is a 5-methylcytosine.

In one embodiment, Compound A is administered as a free acid. In another embodiment, Compound A is administered as a pharmaceutically acceptable salt thereof.

In some embodiments, the modified oligonucleotide consists of 20 linked nucleosides and having a nucleobase sequence of SEQ ID NO: 1. In some embodiments, the modified oligonucleotide is bepirovirsen. In some embodiments, bepirovirsen is administered as a free acid, a pharmaceutically acceptable salt thereof (e.g., a sodium salt), or a combination thereof. In some embodiments, bepirovirsen is administered as a free acid. In some embodiments, bepirovirsen is administered as a pharmaceutically acceptable salt thereof (e.g., a sodium salt). In some embodiments, bepirovirsen is administered as a combination of a free acid and a sodium salt.

In some embodiments, the methods for treating an HBV infection in a subject in need thereof comprises administering to the subject a therapeutically effective amount of Compound A and a therapeutically effective amount of bepirovirsen. The therapeutically effective amount of bepirovirsen is calculated based on the amount of bepirovirsen free acid. The subject may be a human.

In one embodiment, the present disclosure provides a method for treating chronic hepatitis B (CHB) in a human in need thereof, the method comprising administering to the subject a therapeutically effective amount of Compound A and a therapeutically effective amount of bepirovirsen.

In one embodiment, the subject is on stable nucleos(t)ide analogue (NA) therapy (e.g., tenofovir disoproxil, tenofovir alafenamide, or entecavir). “Stable” is defined as no changes to the nucleos(t)ide regimen for at least 6 months prior to the treatment and with no planned changes to the regimen for the duration of the treatment. In some embodiments, the NA therapy is lamivudine, adefovir, adefovir dipivoxil, telbivudine, entecavir, tenofovir, tenofovir disoproxil fumarate (TDF), or tenofovir alafenamide (TAF), or a pharmaceutically acceptable salt thereof. In some embodiments, the NA therapy is entecavir, tenofovir, tenofovir disoproxil fumarate, or tenofovir alafenamide. In some embodiments, the NA therapy is entecavir. In some embodiments, the NA therapy is tenofovir. In some embodiments, the NA therapy is tenofovir disoproxil fumarate. In some embodiments, the NA therapy is tenofovir alafenamide.

In another embodiment, the subject is not on NA therapy. In some embodiments, the subject is treatment-naïve.

In some embodiments, Compound A is administered orally. In one embodiment, Compound A is administered as a capsule. In one embodiment, Compound A is administered as a tablet. In some embodiments, Compound A is administered to a subject under fasted conditions. In some embodiments, Compound A is administered to a subject under fed conditions. In some embodiments, Compound A is administered for 1 week, 2 weeks, 3 weeks, or 4 weeks. In some embodiments, Compound A is administered for 4 weeks, 8 weeks, or 12 weeks. In one embodiment, Compound A is administered for 4 weeks.

In some embodiments, the modified oligonucleotide is administered by subcutaneous injection. In some embodiments, the modified oligonucleotide is administered by subcutaneous injection in an aqueous solution. In some embodiments, the modified oligonucleotide is administered at a dose of about 150 mg or 300 mg once weekly. In one embodiment, the modified oligonucleotide is administered at a dose of about 150 mg once weekly. In another embodiment, the modified oligonucleotide is administered at a dose of about 300 mg once weekly. In some embodiments, the modified oligonucleotide is administered weekly with additional loading doses in the first two weeks on Day 4 and Day 11. In another embodiment, the modified oligonucleotide is administered at a dose of about 300 mg once weekly, with additional loading doses on Day 4 and Day 11.

In some embodiments, the modified oligonucleotide is administered for about 12-24 weeks. In some embodiments, the modified oligonucleotide is administered for 12 weeks, 14 weeks, 16 weeks, 18 weeks, 20 weeks, 22 weeks, or 24 weeks. In one embodiment, the modified oligonucleotide is administered for 12 weeks. In one embodiment, the modified oligonucleotide is administered for 24 weeks. In one embodiment, the modified oligonucleotide is bepirovirsen and is administered for 12 weeks or 24 weeks, with additional loading doses on Day 4 and Day 11.

In some embodiments, Compound A and the modified oligonucleotide are administered concomitantly. In some embodiments, Compound A and the modified oligonucleotide are administered concomitantly for a first treatment period. In some embodiments, the first treatment period is 2 to 12 weeks, for example, 2 weeks, 3 weeks, 4 weeks, 6 weeks, 8 weeks, 10 weeks, or 12 weeks. In some embodiments, the first treatment period is 4 weeks, 5 weeks, 6 weeks, 7 weeks, 8 weeks, 9 weeks, 10 weeks, 11 weeks, or 12 weeks. In some embodiments, the first treatment period is 4 to 12 weeks. In one embodiment, Compound A and bepirovirsen are administered concomitantly for a first treatment period (e.g. 4 weeks, 8 weeks, or 12 weeks). In one embodiment, Compound A and bepirovirsen are administered concomitantly for 4 weeks. In one embodiment, Compound A and bepirovirsen are administered concomitantly for 8 weeks. In one embodiment, Compound A and bepirovirsen are administered concomitantly for 12 weeks.

In some embodiments, after the concomitant administration for a first treatment period, the modified oligonucleotide is administered alone for a second treatment period. In some embodiments, the second treatment period is 8 to 20 weeks. In some embodiments, the second treatment period is 8 to 48 weeks. In some embodiments, the second treatment period is 8 weeks, 9 weeks, 10 weeks, 11 weeks, 12 weeks, 13 weeks, 14 weeks, 15 weeks, 16 weeks, 17 weeks, 18 weeks, 19 weeks, or 20 weeks. In one embodiment, the second treatment period is 8 weeks. In one embodiment, the second treatment period is 20 weeks.

In other embodiments, after the concomitant administration for a first treatment period, Compound A is administered alone for a second treatment period. In some embodiments, the second treatment period is 4 to 12 weeks. In some embodiments, the second treatment period is 4 weeks, 5 weeks, 6 weeks, 7 weeks, 8 weeks, 9 weeks, 10 weeks, 11 weeks, or 12 weeks.

In some embodiments, Compound A and the modified oligonucleotide are administered concomitantly for a first treatment period, and then the modified oligonucleotide is administered alone for a second treatment period. In some embodiments, the first treatment period is 4 weeks and the second treatment period is 8 to 20 weeks. In some embodiments, Compound A and bepirovirsen are administered concomitantly for 4 weeks and then bepirovirsen is administered alone for 8 weeks, 9 weeks, 10 weeks, 11 weeks, 12 weeks, 13 weeks, 14 weeks, 15 weeks, 16 weeks, 17 weeks, 18 weeks, 19 weeks, or 20 weeks. In one embodiment, Compound A and bepirovirsen are administered concomitantly for 4 weeks and then bepirovirsen is administered alone for 8 weeks. In one embodiment, Compound A and bepirovirsen are administered concomitantly for 4 weeks and then bepirovirsen is administered alone for 20 weeks.

In some embodiments, Compound A and the modified oligonucleotide are administered concomitantly for a first treatment period, and then Compound A is administered alone for a second treatment period. In some embodiments, the first treatment period is 12 weeks and the second treatment period is 4 to 12 weeks. In some embodiments, Compound A and bepirovirsen are administered concomitantly for 12 weeks and then Compound A is administered alone for 4 weeks, 5 weeks, 6 weeks, 7 weeks, 8 weeks, 9 weeks, 10 weeks, 11 weeks, or 12 weeks.

The present disclosure provides that the amount of Compound A and/or the single-stranded oligonucleotide administered to a subject as set forth herein is a therapeutically effective amount. In some embodiments, Compound A is administered twice a day. In some embodiments, the amount of Compound A administered is between about 0.02-20 mg per dose, or about 0.02 mg, 0.05 mg, 0.1 mg, 0.3 mg, 0.5 mg, 1 mg, 3 mg, 10 mg, or 20 mg per dose, or in a range between any two of the preceding values (e.g. 0.02-0.05 mg, 0.02-0.1 mg, 0.02-0.3 mg, 0.02-1 mg, 0.02-3 mg, 0.02-10 mg, 0.05-0.1 mg, 0.05-0.3 mg, 0.05-1 mg, 0.05-3 mg, 0.05-10 mg, 0.1-0.3 mg, 0.1-1 mg, 0.1-3 mg, 0.1-10 mg, 0.3-1 mg, 0.3-3 mg, 0.3-10 mg, 0.5-1 mg, 0.5-3 mg, 0.5-10 mg, 1-3 mg, 1-10 mg, or 3-10 mg). In some embodiments, Compound A is administered at about 0.5 mg, 1 mg, 2 mg, 3 mg, 4 mg, 5 mg, 6 mg, 7 mg, 8 mg, 9 mg, or 10 mg per dose, or in a range between any two of the preceding values, for example, about 0.5-1 mg, about 0.5-2 mg, about 1-10 mg, about 2-6 mg, or about 3-5 mg per dose. In some embodiments, Compound A is administered at about 0.5 mg, 1 mg, 3 mg, or 10 mg per dose. In some embodiments, Compound A is administered at about 0.5 mg per dose. In some embodiments, Compound A is administered at about 1 mg per dose. In some embodiments, Compound A is administered at about 3 mg per dose.

In some embodiments, Compound A is administered at a dose of about 1 to 10 mg or about 1 to 20 mg twice a day. In some embodiments, Compound A is administered at a dose of about 3 mg twice a day. In some embodiments, Compound A is administered at a dose of about 0.5 mg or about 1 mg twice a day. In some embodiments, Compound A is administered at a dose of about 0.5 mg twice a day. In some embodiments, Compound A is administered at a dose of about 1 mg twice a day.

In certain embodiments, administration of Compound A and the modified oligonucleotide decreases HBV RNA levels, HBV DNA levels, HBV protein levels, HBsAg levels, or HBeAg levels in the blood by at least 15, 20, 25, 30, 35, 40, 45, 50, 55, 60, 65, 70, 75, 80, 85, 90, 95 or 99%. In some embodiments, administration of Compound A and bepirovirsen decreases serum HBV DNA levels and/or serum HBsAg levels by at least 15, 20, 25, 30, 35, 40, 45, 50, 55, 60, 65, 70, 75, 80, 85, 90, 95 or 99%.

In some embodiments, the hepatitis B virus infection is caused by any of the human geographical genotypes: A (Northwest Europe, North America, Central America); B (Indonesia, China, Vietnam); C (East Asia, Korea, China, Japan, Polynesia, Vietnam); D (Mediterranean area, Middle East, India); E (Africa); F (Native Americans, Polynesia); G (United States, France); or H (Central America). In some embodiments, the subject has chronic hepatitis B (CHB).

In some embodiments, the subject achieves seroclearance of hepatitis B surface antigen (HBsAg) at the end of the treatment. In some embodiments, the subject maintains seroclearance of HBsAg 24 weeks after the end of the treatment. “The end of the treatment” refers to administration of the final dose of Compound A or the final dose of the modified oligonucleotide for the methods described herein, whichever is later.

In some embodiments, the subject achieves seroclearance of HBV DNA at the end of the treatment. In some embodiments, the subject maintains seroclearance of HBV DNA 24 weeks after the end of the treatment.

In some embodiments, the subject achieves seroclearance of HBsAg at the end of the treatment. In some embodiments, the subject maintains seroclearance of HBsAg 24 weeks after the end of the treatment.

In some embodiments, the subject achieves seroclearance of HBsAg and HBV DNA at the end of the treatment. In some embodiments, the subject maintains seroclearance of HBsAg and HBV DNA 24 weeks after the end of the treatment.

The methods described herein provide a higher response rate for the subjects receiving the combination therapy (Compound A and bepirovirsen) to achieve seroclearance of HBV DNA at the end of the treatment or 24 weeks after the end of the treatment, as compared to those receiving a monotherapy (Compound A or bepirovirsen). In some embodiments, the response rate for the subjects receiving the combination therapy is at least 5%, 6%, 7%, 8%, 9%, 10%, 11%, 12%, 13%, 14%, or 15% higher than those receiving a monotherapy.

The methods described herein also provide a higher response rate for the subjects receiving the combination therapy (Compound A and bepirovirsen) to achieve seroclearance of HBsAg at the end of the treatment or 24 weeks after the end of the treatment, as compared to those receiving a monotherapy (Compound A or bepirovirsen). In some embodiments, the response rate for the subjects receiving the combination therapy is at least 5%, 6%, 7%, 8%, 9%, 10%, 11%, 12%, 13%, 14%, or 15% higher than those receiving a monotherapy.

The methods described herein further provide a higher response rate for the subjects receiving the combination therapy (Compound A and bepirovirsen) to achieve seroclearance of HBsAg and HBV DNA at the end of the treatment or 24 weeks after the end of the treatment, as compared to those receiving a monotherapy (Compound A or bepirovirsen). In some embodiments, the response rate for the subjects receiving the combination therapy is at least 5%, 6%, 7%, 8%, 9%, 10%, 11%, 12%, 13%, 14%, or 15% higher than those receiving a monotherapy.

In some embodiments, the subject achieves a reduction of at least 1, at least 1.5, at least 2, at least 2.5, or at least 3 log 10 IU/mL in HBsAg levels from baseline at the end of the treatment. In some embodiments, the subject maintains a reduction of at least 1, at least 1.5, at least 2, at least 2.5, or at least 3 log 10 IU/mL in HBsAg levels from baseline 24 weeks after the end of the treatment.

In another aspect, the present disclosure provides a combination for use in the treatment of an HBV infection, comprising Compound A having the structure:

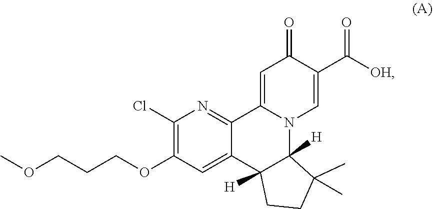 [SMILES: COCCCOc1cc2c(nc1Cl)-c1cc(=O)c(C(=O)O)cn1[C@H]1[C@@H]2CCC1(C)C]

or a pharmaceutically acceptable salt thereof, and
a single-stranded modified oligonucleotide comprising 20 linked nucleosides and having a nucleobase sequence of SEQ ID NO:1, wherein the modified oligonucleotide comprises:
a gap segment consisting of ten linked deoxynucleosides,
a 5′ wing segment consisting of 5 linked nucleosides, and
a 3′ wing segment consisting of 5 linked nucleosides,
   wherein the gap segment is positioned between the 5′ wing segment and the 3′ wing segment, wherein each nucleoside of each wing segment comprises a 2′-O-methoxyethyl sugar, wherein each internucleoside linkage is a phosphorothioate linkage, and wherein each cytosine is a 5-methylcytosine.

In another aspect, the present disclosure relates to use of Compound A in the manufacture of a medicament for the treatment of an HBV infection, wherein Compound A is to be administered with a single-stranded modified oligonucleotide comprising 20 linked nucleosides and having a nucleobase sequence of SEQ ID NO: 1, wherein the modified oligonucleotide comprises:
a gap segment consisting of ten linked deoxynucleosides,
a 5′ wing segment consisting of 5 linked nucleosides, and
a 3′ wing segment consisting of 5 linked nucleosides,
   wherein the gap segment is positioned between the 5′ wing segment and the 3′ wing segment, wherein each nucleoside of each wing segment comprises a 2′-O-methoxyethyl sugar, wherein each internucleoside linkage is a phosphorothioate linkage, and wherein each cytosine is a 5-methylcytosine.

In another aspect, the present disclosure relates to use of a single-stranded modified oligonucleotide in the manufacture of a medicament for the treatment of an HBV infection,
wherein the modified oligonucleotide comprises 20 linked nucleosides and has a nucleobase sequence of SEQ ID NO:1, and comprises:
a gap segment consisting of ten linked deoxynucleosides,
a 5′ wing segment consisting of 5 linked nucleosides, and
a 3′ wing segment consisting of 5 linked nucleosides,
   wherein the gap segment is positioned between the 5′ wing segment and the 3′ wing segment, wherein each nucleoside of each wing segment comprises a 2′-O-methoxyethyl sugar, wherein each internucleoside linkage is a phosphorothioate linkage, and wherein each cytosine is a 5-methylcytosine.

In another aspect, the present disclosure relates to use of a combination of Compound A and a single-stranded modified oligonucleotide in the manufacture of a medicament for the treatment of an HBV infection,
wherein the modified oligonucleotide comprises 20 linked nucleosides and has a nucleobase sequence of SEQ ID NO:1, and comprises:
a gap segment consisting of ten linked deoxynucleosides,
a 5′ wing segment consisting of 5 linked nucleosides, and
a 3′ wing segment consisting of 5 linked nucleosides,
   wherein the gap segment is positioned between the 5′ wing segment and the 3′ wing segment, wherein each nucleoside of each wing segment comprises a 2′-O-methoxyethyl sugar, wherein each internucleoside linkage is a phosphorothioate linkage, and wherein each cytosine is a 5-methylcytosine.

In some embodiments, the combination of Compound A and the modified oligonucleotide (e.g. bepirovirsen) is for use in the treatment of chronic hepatitis B (CHB) in a human in need thereof.

In some embodiments, the combination of Compound A and the modified oligonucleotide (e.g. bepirovirsen) is for use in the manufacture of a medicament for the treatment of CHB in a human in need thereof.

Doses and dosage intervals recited above in relation to methods of the invention are also appropriate for medical uses of the invention.

### Combinations

The present disclosure also provides pharmaceutical combinations for treating a hepatitis B virus (HBV) infection. In some embodiments, the HBV infection is chronic hepatitis B (CHB). In addition to the features described below, the combinations disclosed herein may be used in any of the preceding methods.

The combination comprises two compositions: a first composition comprising a first pharmaceutical component, and a second composition comprising a second pharmaceutical component. For example, in one embodiment, the first composition comprises a PAPD5/7 inhibitor such as Compound A, and/or the second composition comprises an antisense oligonucleotide such as bepirovirsen.

In some embodiments, the combination comprises a PAPD5/7 inhibitor and an antisense oligonucleotide. In particular, the combination comprises a first composition comprising a PAPD5/7 inhibitor, and a second composition comprising an antisense oligonucleotide. In these embodiments, the combination may be for use in the treatment of an HBV infection in a subject in need thereof, wherein the treatment comprises administering to the subject a therapeutically effective amount of a PAPD5/7 inhibitor and an antisense oligonucleotide.

In some embodiments, the combination comprises Compound A having the structure:

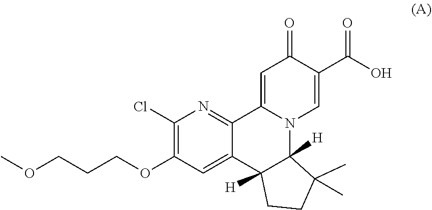 [SMILES: COCCCOc1cc2c(nc1Cl)-c1cc(=O)c(C(=O)O)cn1[C@H]1[C@@H]2CCC1(C)C]

or a pharmaceutically acceptable salt thereof.

In another embodiment, the combination comprises Compound A and a single-stranded modified oligonucleotide comprising 20 linked nucleosides having the nucleobase sequence:

(SEQ ID NO: 1)
5′-GCAGAGGTGAAGCGAAGTGC-3′,

wherein the modified oligonucleotide comprises:
   a gap segment consisting of ten linked deoxynucleosides,
   a 5′ wing segment consisting of 5 linked nucleosides, and
   a 3′ wing segment consisting of 5 linked nucleosides,
wherein the gap segment is positioned between the 5′ wing segment and the 3′ wing segment, wherein each nucleoside of each wing segment comprises a 2′-O-methoxyethyl sugar, wherein each internucleoside linkage is a phosphorothioate linkage, and wherein each cytosine is a 5-methylcytosine.

In one embodiment, Compound A is a free acid. In another embodiment, Compound A is a pharmaceutically acceptable salt thereof.

In some embodiments, the modified oligonucleotide consists of 20 linked nucleosides having a nucleobase sequence of SEQ ID NO: 1. In some embodiments, the modified oligonucleotide is bepirovirsen. In some embodiments, bepirovirsen is administered as a free acid, a pharmaceutically acceptable salt thereof (e.g., a sodium salt), or a combination thereof. In some embodiments, bepirovirsen is administered as a free acid. In some embodiments, bepirovirsen is administered as a pharmaceutically acceptable salt thereof (e.g., a sodium salt). In some embodiments, bepirovirsen is administered as a combination of a free acid and a sodium salt.

In some embodiments, the combination is for use in a method of treating an HBV infection in a subject in need thereof, wherein the method comprises administering to the subject a therapeutically effective amount of Compound A and a therapeutically effective amount of bepirovirsen.

In one embodiment, the present disclosure provides a combination for use in a method of treating chronic hepatitis B (CHB) in a human in need thereof, the method comprising administering to the human a therapeutically effective amount of Compound A and a therapeutically effective amount of bepirovirsen.

In one embodiment, the subject is on stable nucleos(t)ide analogue (NA) therapy (e.g., tenofovir disoproxil, tenofovir alafenamide, or entecavir). In some embodiments, the NA therapy is lamivudine, adefovir, adefovir dipivoxil, telbivudine, entecavir, tenofovir, tenofovir disoproxil fumarate (TDF), or tenofovir alafenamide (TAF), or a pharmaceutically acceptable salt thereof. In some embodiments, the NA therapy is entecavir, tenofovir, tenofovir disoproxil fumarate, or tenofovir alafenamide. In some embodiments, the NA therapy is entecavir. In some embodiments, the NA therapy is tenofovir. In some embodiments, the NA therapy is tenofovir disoproxil fumarate. In some embodiments, the NA therapy is tenofovir alafenamide.

In another embodiment, the subject is not on NA therapy prior to administration of Compound A or the modified oligonucleotide. In some embodiments, the subject is treatment-naïve.

In some embodiments, Compound A is for oral administration. In one embodiment, Compound A is formulated as a capsule. In one embodiment, Compound A is formulated as a tablet.

In some embodiments, the modified oligonucleotide is formulated for delivery by subcutaneous injection. In some embodiments, the modified oligonucleotide is in an aqueous solution. In some embodiments, the combination comprises 150 mg or 300 mg of the modified oligonucleotide.

In some embodiments, the combination is for use in a method wherein Compound A and the modified oligonucleotide are administered concomitantly. In some embodiments, this concomitant administration takes place for a first treatment period. In some embodiments, the first treatment period is 2 to 12 weeks, for example, 2 weeks, 3 weeks, 4 weeks, 5 weeks, 6 weeks, 7 weeks, 8 weeks, 9 weeks, 10 weeks, 11, weeks, or 12 weeks. In some embodiments, the first treatment period is 4 weeks, 5 weeks, 6 weeks, 7 weeks, 8 weeks, 9 weeks, 10 weeks, 11 weeks, or 12 weeks. In one embodiment, the first treatment period is 4 weeks. In one embodiment, the combination comprises Compound A and bepirovirsen for use in a method wherein they are administered concomitantly for a first treatment period (e.g. 4 weeks, 8 weeks, or 12 weeks). In one embodiment, the combination comprises Compound A and bepirovirsen for use in a method wherein they are administered concomitantly for 4 weeks.

The present disclosure provides a combination comprising therapeutically effective amounts of Compound A and the single-stranded oligonucleotide. In some embodiments, the combination comprises 0.02-20 mg of Compound A, or 0.02 mg, 0.05 mg, 0.1 mg, 0.3 mg, 0.5 mg, 1 mg, 3 mg, 10 mg, or 20 mg of Compound A, or an amount in a range between any two of the preceding values (e.g. 0.02-0.05 mg, 0.02-0.1 mg, 0.02-0.3 mg, 0.02-1 mg, 0.02-3 mg, 0.02-10 mg, 0.05-0.1 mg, 0.05-0.3 mg, 0.05-1 mg, 0.05-3 mg, 0.05-10 mg, 0.1-0.3 mg, 0.1-1 mg, 0.1-3 mg, 0.1-10 mg, 0.3-1 mg, 0.3-3 mg, 0.3-10 mg, 0.5-1 mg, 0.5-3 mg, 0.5-10 mg, 1-3 mg, 1-10 mg, or 3-10 mg). In some embodiments, the combination comprises 0.5 mg, 1 mg, 2 mg, 3 mg, 4 mg, 5 mg, 6 mg, 7 mg, 8 mg, 9 mg, or 10 mg of Compound A, or an amount between any two of the preceding values, for example, about 0.5-1 mg, 1-10 mg, 2-6 mg, or about 3-5 mg. In some embodiments, the combination comprises 0.5 mg, 1 mg, 3 mg, or 10 mg of Compound A. In some embodiments, the combination comprises 3 mg of Compound A. In some embodiments, the combination comprises 0.5 mg of Compound A. In some embodiments, the combination comprises 1 mg of Compound A.

In one embodiment, the combination comprises 150 mg of the modified oligonucleotide and 3 mg of Compound A. In another embodiment, the combination comprises 300 mg of the modified oligonucleotide and 3 mg of Compound A. In one embodiment, the combination comprises 150 mg of the modified oligonucleotide and 0.5-1 mg of Compound A. In another embodiment, the combination comprises 300 mg of the modified oligonucleotide and 0.5-1 mg of Compound A. In one embodiment, the combination comprises 150 mg of the modified oligonucleotide and 0.5 mg of Compound A. In another embodiment, the combination comprises 300 mg of the modified oligonucleotide and 0.5 mg of Compound A. In one embodiment, the combination comprises 150 mg of the modified oligonucleotide and 1 mg of Compound A. In another embodiment, the combination comprises 300 mg of the modified oligonucleotide and 1 mg of Compound A.

### Kits

In an aspect of the invention, the active pharmaceutical agents disclosed herein, in particular in the form of pharmaceutical compositions, are included in a kit with instructions for use. In an embodiment, the kit comprises an antisense oligonucleotide targeted to HBV, i.e., the modified oligonucleotide as disclosed herein, and a PAPD5/7 inhibitor, as disclosed herein, in separate containers. In an embodiment, the kit comprises bepirovirsen. In an embodiment, the kit comprises Compound A or a pharmaceutically acceptable salt thereof. In an embodiment, the kit comprises bepirovirsen. and Compound A or a pharmaceutically acceptable salt thereof.

For convenience, the kit may comprise the active pharmaceutical agents in predetermined amounts with instructions for use. In an embodiment, the predetermined amounts are as disclosed in the Combinations section above. In an embodiment, the kit comprises the 300 mg of modified oligonucleotide. In an embodiment, the kit comprises 150 mg of modified oligonucleotide.

In an embodiment, the kit comprises Compound A formulated for oral administration. In an embodiment, the kit comprises one or more capsules or tablets comprising Compound A. In an embodiment, the kit comprises one or more capsules or tablets comprising 0.02-20 mg of Compound A. In an embodiment, each capsule or tablet comprises 0.5-1 mg of compound A. In an embodiment, each capsule or tablet comprises 0.5 mg of Compound A. In an embodiment, each capsule or tablet comprises 1 mg of Compound A. In an embodiment, Compound A is formulated as a tablet. In an embodiment, Compound A is formulated in a capsule.

The kit may also include a device to be used for administration of a pharmaceutical composition.

In another embodiment, the kit comprises other medicaments.

### EXAMPLES

While aspects of the disclosure presented herein have been described more particularly in accordance with some embodiments, the following examples, which highlight certain features and properties of the exemplary embodiments of the disclosure described herein, serve only to illustrate the disclosure described herein and are not intended to limit the same.

### Example 1: In Vitro Antiviral Activity of Compound A

The potency (pIC₅₀) of Compound A for reduction of HBV HBsAg was investigated in primary human hepatocytes (PHH) from three donors. HBV infected PHH samples were treated with Compound A for 21 days and secreted HBsAg was measured using an ELISA assay to determine the % inhibition and average potency of the replicates (two per PHH donor). Compound A showed a similar potency in HBsAg reduction across the three different PHH donors with an average pIC50 of 9.08. Visual examination showed no detected sign of cell toxicity.

### Example 2: Toxicity Evaluation of PAPD5/7 Inhibitors

Studies were conducted to evaluate toxicity, including additional endpoints investigating nerve toxicity of Compound A after 28 days of dosing followed by a 6 week off dose period in rats and monkeys. No adverse effect doses were established for Compound A in both species. Another PAPD5/7 inhibitor RG7834 (structure shown below) was terminated in Phase 1 clinical trials due to toxicity demonstrated preclinically where a no adverse effect dose could not be established.

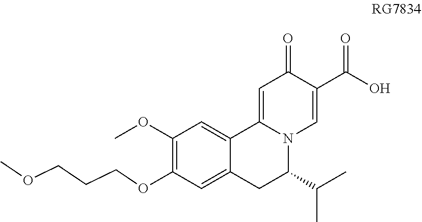 [SMILES: COCCCOc1cc2c(cc1OC)-c1cc(=O)c(C(=O)O)cn1[C@H](C(C)C)C2]

### Example 3: Comparison of Antiviral Activity of PAPD5/7 Inhibitors

The potency of Compound A, RG7834, and Compound B (structure shown below) for reducing HBV HBsAg was investigated in HepAD38 cell line. Compound A showed higher potency as compared to RG7834 and Compound B (Table 1).

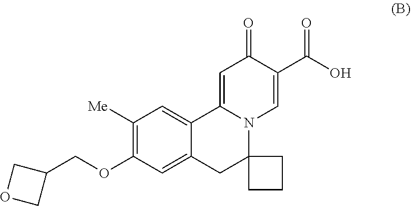 [SMILES: Cc1cc2c(cc1OCC1COC1)CC1(CCC1)n1cc(C(=O)O)c(=O)cc1-2]

HepAD38 cells were maintained in collagen-coated flasks in cell culture medium (DMEM/F12 containing 10% fetal bovine serum (FBS), GlutaMax-1, penicillin/streptomycin, non-essential amino acids, Na pyruvate, 250 μg/mL geneticin, and 1 μg/mL doxycycline). Compound solutions were prepared in DMSO and compounds were serially diluted for final concentrations of 4000, 1000, 250, 62.5, 15.6, 3.91, 0.977, 0.244, 0.061, and 0.015 nM. The cells were then trypsinized and the cells were plated at 10,000 cells per well. Plates were incubated at 37° C., 5% CO₂ for 4 days. Media was replaced with new media with compound treatment. The plates were then incubated at 37° C., 5% CO₂ for an additional 3 days for a total treatment time of 7 days. For antiviral response, HBsAg was measured using the HBsAg ELISA kit (International Immuno-diagnostics) with instructions provided. 100 μL of cell media samples was used for ELISA. The absorbance was read on the SPECTRAMAX 384 plate reader (Molecular Devices) at 450 nm. For cell toxicity, the cells were used for CELLTITER-GLO Luminescent Cell Viability Assay reagent (Promega). The luminescence was read on Envision Multilabel Reader (Perkin Elmer). The data was analyzed compared to the DMSO control.

% inhibition=(1−(unknown/high control))*100

The average % inhibition values from duplicate assay plates were then plotted in GraphPad Prism to determine one EC value: Four-parameter logistic curve with equation Y=Bottom+(Top-Bottom)/(1+10\hat{ }((Log EC50−X)*Hillslope))

TABLE 1
Antiviral Activity of PAPD5/7 Inhibitors

| | Compound A | RG7834 | Compound B |
|---|---|---|---|
| HBsAg (EC₅₀) | 0.3 nM | 2 nM | 3 nM |

### Example 4: Comparison of Biochemical Enzyme Inhibition of PAPD5/7 Inhibitors

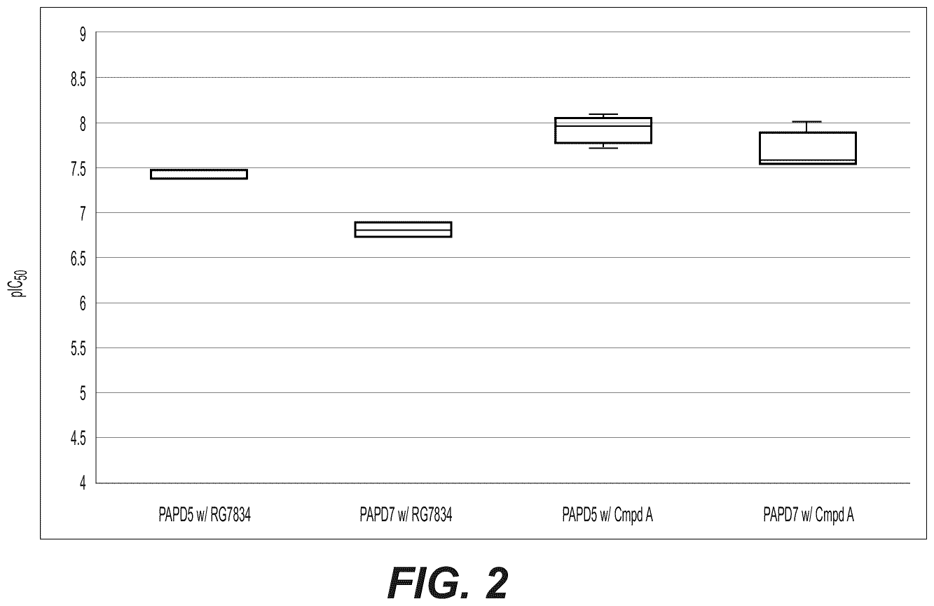

Dose-response studies were conducted for Compound A and RG7834 in the PAPD5 and PAPD7 A15 RNA SPA formats. Four different batches of Compound A and two different batches of RG7834 were used in both assays. Reactions contained 5 uM ATP, 0.0025 uCi/uL 3H-ATP, 50 nM A15 RNA, 10 nM enzyme in assay buffer (20 mM Tris 7.5, 3 mM CHAPS, 0.05% dBSA, 10 mM MgCl₂, 1 mM DTT, 25 mM KCl). Reaction product was detected using SPA bead mix, final concentrations were 1 mg/mL PEI-PS, 1.25 mM ATP, 11.25 mM EDTA. End point signal detection correlated with slopes of reaction time courses. Curve fitting was performed using 4-parameter fitting in ActivityBase software. Compound A average pIC50 values in the PAPD5 and PAPD7 assays were 7.9 and 7.7, respectively. The data shows that Compound A was more potent than RG7834 by >0.5 log (see FIG. 2).

### Example 5: In Vivo Antiviral Activity in AAV-HBV Mouse Models

### A. Mono-Treatment

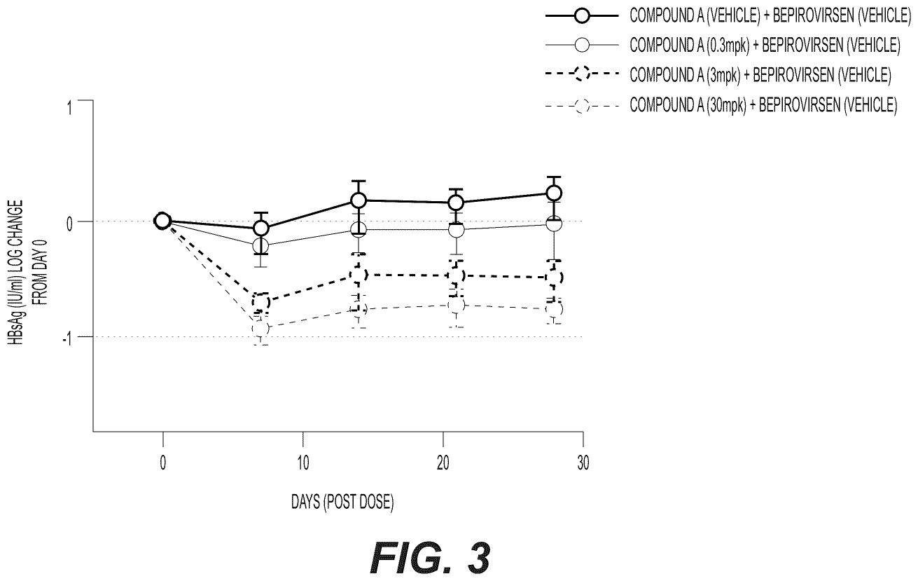

After 28 days of infection with AAV-HBV, mono-treatment of the infected male C57BL/6 mice with either Compound A or bepirovirsen was initiated. At the combined vehicle and lowest dose of Compound A (0.3 mg/kg), there was minimal reduction in HBsAg over the course of treatment. Animals receiving either the mid (3 mg/kg) or high (30 mg/kg) doses of Compound A demonstrated statistically significant HBsAg reduction compared to the combined vehicle group. HBsAg reduction for the 3 and 30 mg/kg Compound A dose groups was close to 1 log and 1 log, respectively, within one week of treatment and reduction levels were sustained throughout the duration of the treatment period. Following one week off-treatment, HBsAg levels rebounded to baseline. In measuring the effect on levels of HBeAg, there was a dose dependent reduction during treatment with Compound A that stabilized after day 7 and rebounded within one week following the end of treatment. The maximum observed reduction in HBeAg was 0.5 log at 30 mg/kg Compound A. There was a small reduction (0.26 to 0.40 log) in HBV DNA at the 3 and 30 mg/kg Compound A dose levels when compared to the combined vehicle group. The changes in HBsAg due to treatment with Compound A are presented in FIG. 3.

At treatments of either 20 mg/kg or 40 mg/kg of bepirovirsen, maximum HBsAg reduction of over 1 log and 2 logs, respectively, was achieved two weeks following the start of treatment. Return to baseline was gradual through to the end of study for the 20 mg/kg bepirovirsen group, while the 40 mg/kg bepirovirsen group had reduced HBsAg at the end of study. Reduction of HBeAg in the 20 mg/kg and 40 mg/kg bepirovirsen groups stabilized within two weeks of treatment at 1.35 log and 1.85 log, respectively, when compared to the combined vehicle group. Following end of treatment, HBeAg rebounded slowly and remained reduced at 0.34 log and 0.80 log in the 20 mg/kg bepirovirsen and 40 mg/kg dose groups, respectively. All treatments were well tolerated based on body weight profiles.

### B. Concurrent Treatment

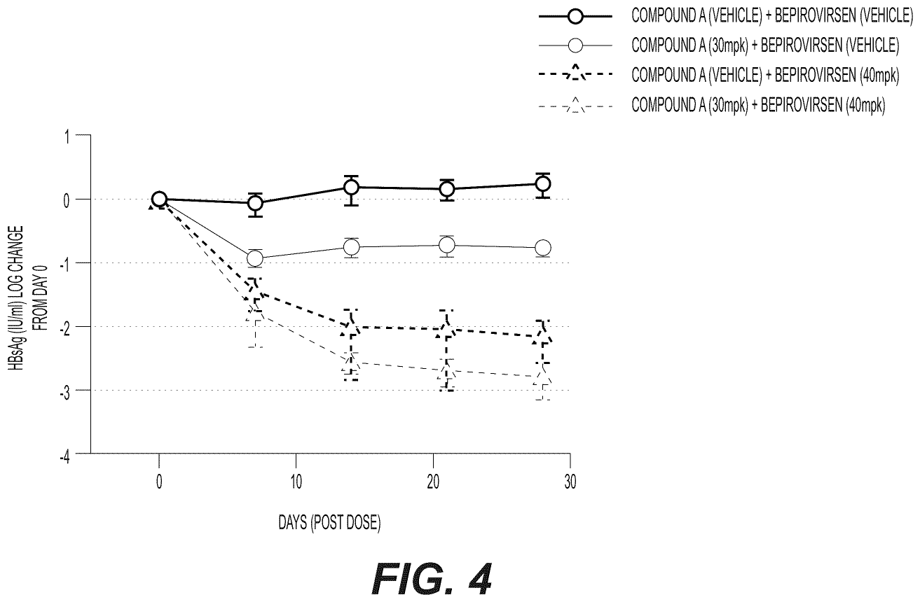

Following 28 days of infection with AAV-HBV, concurrent treatment of the infected male C57BL/6 mice with Compound A and bepirovirsen was initiated. For the groups which received both compounds, an enhanced effect on reduction of HBsAg for 3 mg/kg or 30 mg/kg Compound A with 20 mg/kg or 40 mg/kg bepirovirsen was observed. The maximum observed reduction in HBsAg was more than 2 log and nearly 3 log for the 30 mg/kg Compound A+20 mg/kg bepirovirsen and 30 mg/kg Compound A+40 mg/kg bepirovirsen groups, respectively. The effect of the concurrent treatment on levels of HBsAg at the highest dose of Compound A with and without bepirovirsen is presented in FIG. 4. The effects on reduction of HBeAg was measured, and HBeAg was reduced by 0.15 to 0.44 log in the Compound A 3 or 30 mg/kg+bepirovirsen 20 or 40 mg/kg dose groups. Rebound of HBeAg in these combination groups occurred similarly to the mono-therapy dose groups for bepirovirsen. There was a strong reduction in HBV DNA of more than 2.5 log in all bepirovirsen dose groups (with or without Compound A) that stabilized by day 14 of treatment. At study end, HBV DNA remained reduced by 0.84 to 1.35 log for groups receiving 20 mg/kg bepirovirsen and by 1.72 to 2.01 log for groups receiving 40 mg/kg bepirovirsen when compared to the combined vehicle group. All treatments were well tolerated based on body weight profiles.

### C. Sequential Treatment

After 35 days of infection with AAV-HBV, sequential treatment of the infected male C57BL/6 mice began first with Compound A followed by bepirovirsen. Within one week of treatment with 30 mg/kg Compound A alone, a statistically significant reduction in HBsAg at nearly 1 log was observed when compared to groups receiving the Compound A vehicle, and was stable for the duration (up to 28 days) of the lead-in treatment with Compound A. Within one week off-treatment, HBsAg rebounded to baseline and was indistinguishable from the levels of the combined vehicle group.

Within one week of treatment start with bepirovirsen, dosed at concentrations of either 20 or 40 mg/kg with the Compound A vehicle, reductions in HBsAg was observed, reaching a maximum reduction of over 1 log and nearly 2 logs, respectively, by the second week. This reduction was statistically significant when compared to the combined vehicle group. In the groups dosed initially with Compound A followed by bepirovirsen (at 30 mg/kg Compound A then either 20 or 40 mg/kg bepirovirsen), additional HBsAg reduction relative to the reduction maintained during lead-in Compound A treatment was observed by the second week of bepirovirsen treatment.

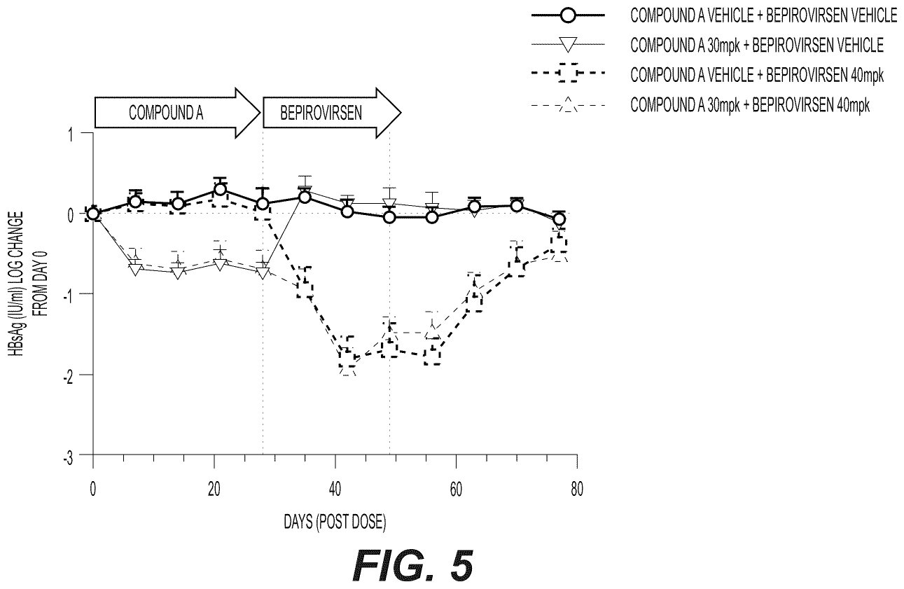

On the last day of treatment with bepirovirsen and one week post the last day, groups treated with 30 mg/kg Compound A followed by either 20 or 40 mg/kg bepirovirsen failed to demonstrate additional HBsAg reduction when compared to groups treated with bepirovirsen alone, suggesting that there is no added benefit of lead-in treatment with Compound A on the HBsAg-reducing effects of bepirovirsen when following a sequential format. Additionally, HBsAg levels rebounded similarly in groups that did or did not receive 30 mg/kg Compound A. All treatment regimens were well tolerated based on body weight profiles. The effect of sequential treatment at the highest doses of Compound A and bepirovirsen is shown in FIG. 5.

### Example 6: Studies for Human Dose Prediction

### A. Human Pharmacokinetics Prediction

A physiologically based PK (PBPK) model with advanced compartmental absorption and transit (ACAT) model linked to the compartmental disposition model using GastroPlus has been established with in silico, in vitro, and in vivo preclinical data. Rat, dog, minipig, and monkey PK data were used to develop the ACAT model and default gastrointestinal physiology settings were used for all species. Overall, the established PBPK model matches the shape of preclinical PK profiles and described the PK parameters AUC and Cmax well, with less than 2-fold difference between predicted and observed values. PK predictions were conducted assuming an average human weight of 70 kg.

The antiviral activity of Compound A was evaluated in in vitro HBV systems: (1) HepAD38, a hepatoma cell line with inducible expression of HBV, (2) primary human hepatocytes (PHHs) infected with HBV, and in vivo in infected male C57BL/6 mice with AAV-HBV. Overall there is a correlation between in vitro potency (HepAD38 & PHHs) and AAV-HBV in vivo model HBsAg inhibition.

The calculated IC90 values based on the above 3 experiments IC50s are shown in Table 2.

TABLE 2
The calculated human total IC90 values based
on the in vitro and in vivo experiments

| | IC50 | Calculated IC90 |
|---|---|---|
| Experiment | ng/mL (nM) | (ng/mL) |
| HepAD38 cells | 0.56 ng/mL (1.3 nM) | 5.04 |
| PHH | 0.95 ng/mL (2.19 nM) | 8.55 |
| In vivo AAV-HBV mice | 0.058 ng/mL (0.13 nM) | 0.522 |

Values are the protein binding adjusted human total concentrations.

### B. Therapeutic Dose Rationale

The minimum therapeutic dose is defined as the dose that provides a Cmin at steady state associated with activity more than the 90% of predicted maximal pharmacologic activity (Cmin>IC90).

Based on the predicted half-life of approximately 13 hours, and consistent with optimized dosing considering safety limits, a dosing frequency of twice daily was selected for Compound A. Moreover, the analyses described in the therapeutic dose rationale supports that this regimen will provide pharmacologic activity over the dosing interval (i.e., at predicted Cmin concentrations at 3 mg BID dosing, the predicted efficacious dose is estimated to provide 91.9%, 87.0%, and 99.1% activity in HepAD38 cells, PHHs, AAV HBV mice experiments, respectively). Twice daily dosing will also minimize the exposure (Cmax) associated with maintaining concentrations above target (EC90), thus ensuring robust safety margins.

### C. Maximum Dose Rationale

The maximum single dose is defined based on the following:
The dose that provides a concentration at 24 hrs post-dose (C24) associated with approximately 99% of predicted maximal pharmacologic activity (pharmacologic activity limit).
The dose to be associated with exposure that is higher than the predicted exposure at highest planned dose in repeat dosing study (on PK basis).

A top dose of Compound A of 20 mg administered as a single dose is proposed for the human trial, based on the predicted human pharmacokinetics including plasma C24 and Cmax data (see Table 3). Considering that the planned highest dose in repeat dosing to be 3× therapeutic BID dose (3×3 mg=9 mg˜10 mg), the top single dose of 20 mg is expected to cover the predicted PK of the planned highest dose (10 mg) in repeat dosing study on PK basis (see Table 3 and Table 4 below).

TABLE 3
Comparison of predicted human pharmacokinetics
for single doses of Compound A

| | | PK Parameters | | | |
|---|---|---|---|---|---|
| Dose | Dose | Cmax | C24 | AUC24 | AUCinf |
| (mg) | (mg/kg) | (ng/mL) | (ng/mL) | (ng*h/mL) | (ng*h/mL) |
| 0.02 | 0.0003 | 0.03 | 0.01 | 0.47 | 0.66 |
| 0.05 | 0.0007 | 0.07 | 0.03 | 1.18 | 1.64 |
| 0.1 | 0.001 | 0.15 | 0.06 | 2.36 | 3.28 |
| 0.3 | 0.004 | 0.44 | 0.17 | 7.06 | 9.93 |
| 1 | 0.014 | 1.46 | 0.56 | 23.49 | 32.87 |
| 3 | 0.043 | 4.32 | 1.68 | 70.06 | 98.07 |
| 10 | 0.143 | 13.68 | 5.60 | 228.36 | 322.43 |
| 20 | 0.286 | 25.55 | 11.22 | 442.22 | 631.39 |

TABLE 4
Comparison of predicted human pharmacokinetics
for repeat dosing of Compound A

| | | | PK Parameters | | |
|---|---|---|---|---|---|
| BID | | | | | Daily |
| Dose | Dose | Cmax | Cmin | AUCss | AUC** |
| (mg) | (mg/kg) | (ng/mL) | (ng/mL) | (ng*h/mL) | (ng*h/mL) |
| 1 | 0.014 | 2.93 | 2.13 | 30.64 | 61.25 |
| 3 | 0.043 | 8.70 | 6.35 | 91.77 | 182.43 |
| 10 | 0.143 | 28.18 | 20.84 | 296.52 | 592.63 |

### Example 7: A 4-Part, Randomized, Double-Blind, Multi-Center, Placebo-Controlled Study to Assess the Safety, Tolerability, PK, and PD of Compound a Monotherapy in Healthy Participants and in CHB Patients; and Compound a in Combination with Bepirovirsen in CHB Patients

The first two parts of this study will evaluate the safety, tolerability, and pharmacokinetics (PK) of single (Part 1) and repeat doses (Part 2A) of Compound A, and tablet/food effect with single dose (Part 2B) in healthy participants. Part 3 will evaluate the ability of Compound A to lower HBsAg in participants living with chronic hepatitis B infection (PLWCHB). Part 4 will evaluate the safety and tolerability of combination therapy with Compound A and bepirovirsen and the potential to effect sustained virologic response in PLWCHB.

Part 4 is a 12-week, repeat dose single dose level study of Compound A in combination with bepirovirsen in PLWCHB on stable NA therapy who have not participated in Part 3 of the study. Sixty participants will be randomized in a 3:1 ratio (45 participants on active and 15 participants on placebo) to receive either Compound A or placebo as oral tablets twice daily (approximately 12 h dosing intervals) for 28 days. The dose of Compound is to be determined based on the results from Parts 1-3 of this study. In addition, all participants in this cohort will also receive open label bepirovirsen (300 mg subcutaneous [SC], weekly, plus loading dose on Day 4 and Day 11) concomitantly for 28 days. Bepirovirsen dosing will continue for additional 8 weeks after Compound A dosing completes (for a total of 12 weeks). The patients will be monitored in follow-up for 24 weeks after last bepirovirsen dose.

The primary efficacy endpoint is sustained virologic response, which is a composite endpoint defined as HBsAg<LLOQ (0.05 IU/mL) and HBV DNA<LLOQ (20 IU/mL) at the end of bepirovirsen treatment which is sustained for 24 weeks post-bepirovirsen treatment. Serum HBsAg level is measured by a sandwich immunoassay with COBAS HBsAg quant II (Roche). Serum HBV DNA level is measure with COBAS Ampliprep/COBAS Taqman HBV test v.2.0 (Roche). Seroclearance in this trial refers to participants with HBsAg and HBV DNA<LLOQ (with or without the formation of HBs-antibody). Seroconversion refers to participants with HBsAg and HBV DNA<LLOQ plus formation of HBs-antibody. Both terms are used to evaluate efficacy. For the purposes of this study, sustained response is defined as a continuous 24 weeks from end of bepirovirsen treatment during which levels of HBsAg in serum remain less than LLOQ and HBV DNA less than LLOQ.

The objectives and endpoints of Part 3 and Part 4 of this study are listed below.

TABLE 5
Objectives and Endpoints

| Objectives | Endpoints |
|---|---|
| Primary | |
| To assess the safety and tolerability of | Incidence of AEs, SAEs, withdrawals due to AEs |
| oral administration of Compound A | Incidence of clinically significant |
| monotherapy (Part 3) and in combination | laboratory parameters (haematology, |
| with bepirovirsen (Part 4) | clinical chemistry, urinalysis), vital signs, |
| | cardiac parameters (electrocardiogram), |
| | and sensory nerve conduction velocity |
| To evaluate pharmacodynamic (PD) | Maximum reduction of serum HBsAg |
| effect of Compound A monotherapy in | levels from baseline over 6 weeks |
| PLWCHB (Part 3) | (4 weeks on treatment and 2 weeks post- |
| | treatment) |
| To evaluate PD effect of Compound A in | Achieving sustained virologic response |
| combination with bepirovirsen in | (undetectable serum HBV DNA and |
| PLWCHB (Part 4) | HBsAg for 6 consecutive months after |
| | the planned end of treatment of |
| | bepirovirsen) |
| Secondary | |
| To evaluate the PK characteristics of | AUC(0-tau), Cmax, Tmax, and apparent |
| repeat doses of Compound A in | terminal half-life (T½) will be |
| PLWCHB (Part 3) | calculated as data permits |
| To evaluate pharmacodynamic (PD) | ≥0.5x log IU/mL reduction of serum |
| effect of Compound A monotherapy in | HBsAg levels from baseline anytime |
| PLWCHB (Part 3) | during the study (on-treatment and post- |
| | treatment) |
| To evaluate PD effect of Compound A in | HBsAg loss (defined by two consecutive |
| combination with bepirovirsen in | measurements of HBsAg below the limit |
| PLWCHB (Part 4) | of quantification) anytime during the |
| | study (on-treatment and post-treatment) |
| Exploratory | |
| To assess the effect of Compound A on | Quantification of HBV RNA, HBcrAg, |
| HBV-related biomarkers | HBeAg, HBsAb and HBeAb |
| To assess the effect of genotype on | Sequencing of the viral HBV DNA |
| response. | and/or HBV RNA prior to treatment, |
| To explore presence of baseline | during treatment and post treatment visits |
| polymorphisms and emergent mutations | PAPD5/7 target(s) RNA tail sequencing |
| and the association with virological | Soluble immunological biomarkers, as |
| response | determined by (but not limited to) levels |
| To investigate Compound A target | of circulating cytokines and chemokines. |
| engagement in peripheral blood (Part 3 | Markers of immune cell function, as |
| only) | measured by (but not limited to) relative |
| To assess the effect of Compound A on | frequencies of immune cell subsets |
| immunologic biomarkers | among PBMCs, activation status as |
| To describe the relationship(s) between | determined by phenotyping and gene |
| virology biomarkers, and immunological | expression patterns, and functional |
| biomarkers | assays including HBV-specific cytokine |
| | and/or antibody production |

### Example 8: In Vitro Antiviral Efficacy Evaluation of the Combination of Bepirovirsen and Compound A

The combined antiviral effect of bepirovirsen and Compound A are evaluated in vitro using HBV-infected primary human hepatocytes (PHH). PHH are allowed 3 days post plating in collagen coated 96-well plates to establish monolayers and assimilate to in vitro culture, followed by HBV infection at an MOI of 200-500 genome equivalents (GE)/cell. HBV infected (or non-infected control) PHH are treated 7 days after HBV infection, and treatment continues at a predetermined dosing frequency until study termination on Day 21 post treatment/Day 28 post infection. Dosing is performed in a checkerboard matrix layout in which 8 serial dilutions of bepirovirsen and 8 serial dilutions of Compound A are combined. The highest evaluated concentration of each treatment is serially diluted 3, 4, of 5-fold to include concentrations expected to result in 0 and maximal % inhibition. The serial dilution scheme positions the EC50 of each treatment, determined in prior in vitro studies of HBV-infected PHH, near the center of the expected dose response curve. In parallel, monotherapy is investigated as a comparator when combined with vehicle control. Each treatment within a single 96-well plate is tested in singleton; however, each plate is run in replicates of 2-3 to assess assay and biological variability. At timepoints such as 7, 14, and 21 days post treatment, culture supernatant is collected and stored until analysis at −80° C. Levels of secreted HBsAg in culture supernatant serve as the primary efficacy readout of bepirovirsen and Compound A activity. Additional viral endpoints may be assessed to determine the antiviral effect of combining bepirovirsen and Compound A on production of other HBV antigens, HBV RNA, and HBV DNA. Statistical analysis is performed using synergy software to determine whether a synergistic, antagonistic or other effect is observed when bepirovirsen and Compound A are combined.

One skilled in the art will readily appreciate that the present disclosure is well adapted to carry out the objects and obtain the ends and advantages mentioned, as well as those inherent therein. The particular embodiments described herein are intended to be representative and exemplary and are not intended as limitations on the scope of the invention. Changes therein and other uses will be apparent to those skilled in the art which are encompassed within the spirit of the invention as defined by the scope of the claims.

All patent applications, patents, and printed publications cited herein are incorporated herein by reference in the entireties, except for any definitions, subject matter disclaimers or disavowals, and except to the extent that the incorporated material is inconsistent with the express disclosure herein, in which case the language in this disclosure controls.

1. A method for treating chronic hepatitis B in a human in need thereof, the method comprising administering to the subject a therapeutically effective amount of Compound A having the structure: 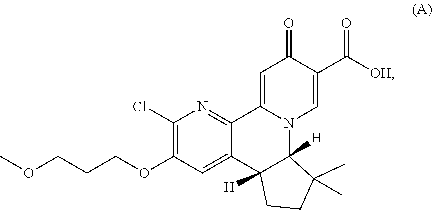 [SMILES: COCCCOc1cc2c(nc1Cl)-c1cc(=O)c(C(=O)O)cn1[C@H]1[C@@H]2CCC1(C)C]
or a pharmaceutically acceptable salt thereof, and
administering to the subject a therapeutically effective amount of a single-stranded modified oligonucleotide comprising 20 linked nucleosides and having a nucleobase sequence of SEQ ID NO: 1, wherein the modified oligonucleotide comprises:
a gap segment consisting of ten linked deoxynucleosides,
a 5′ wing segment consisting of 5 linked nucleosides, and
a 3′ wing segment consisting of 5 linked nucleosides,
wherein the gap segment is positioned between the 5′ wing segment and the 3′ wing segment,
wherein each nucleoside of each wing segment comprises a 2′-O-methoxyethyl sugar,
wherein each internucleoside linkage is a phosphorothioate linkage, and wherein each cytosine is a 5-methylcytosine.

2. The method of claim 1, wherein Compound A is administered as a free acid.

3. The method of claim 1, wherein the single-stranded modified oligonucleotide is bepirovirsen.

4. The method of claim 1, wherein the subject is on stable nucleos(t)ide analogue (NA) therapy.

5. The method of claim 1, wherein the NA therapy is lamivudine, adefovir, adefovir dipivoxil, telbivudine, entecavir, tenofovir, tenofovir disoproxil fumarate, or tenofovir alafenamide, or a pharmaceutically acceptable salt thereof.

6. The method of claim 1, wherein Compound A is administered orally.

7. The method of claim 6, wherein Compound A is administered at a dose of about 0.5 mg or 1 mg twice a day.

8. The method of claim 1, wherein the modified oligonucleotide is administered by subcutaneous injection.

9. The method of claim 1, wherein the modified oligonucleotide is administered at a dose of about 150 mg or 300 mg once weekly.

10. The method of claim 1, wherein Compound A and the modified oligonucleotide are administered concomitantly.

11. The method of claim 10, wherein Compound A and the modified oligonucleotide are administered concomitantly for about 4 weeks.

12. The method of claim 10, wherein the modified oligonucleotide is administered alone for a second treatment period after the concomitant administration.

13. The method of claim 12, wherein the second treatment period is about 8-20 weeks.

14-17. (canceled)
